(19) United States
(12) Patent Application Publication
(10) Pub. No.: US 2016/0100975 A1
(43) Pub. Date: Apr. 14, 2016

(54) PANNUS SUPPORT MEMBER

(71) Applicant: Elizabeth Ann Korzelius, Wilmington, NC (US)

(72) Inventor: Elizabeth Ann Korzelius, Wilmington, NC (US)

(21) Appl. No.: 14/881,832

(22) Filed: Oct. 13, 2015

Related U.S. Application Data
(60) Provisional application No. 62/063,789, filed on Oct. 14, 2014.

(51) Int. Cl. A61F 5/37

(52) U.S. Cl. CPC A61F 5/37

(57) ABSTRACT

A pannus support member is provided. The pannus support member includes a waist band having a first end, a second end, an inner surface and an outer surface. The waist band is securable about an abdomen such that the pannus is contained and lifted by the waist band away from a target area on the abdomen of a patient. The present invention further includes a pair of shoulder straps each having a first end and a second end secured to the waist band. The pair of shoulder straps are adjustable in length, such that the straps apply a lifting force to the waist band to cause the pannus to be maintained in a lifted position away from the target area. A polymer gripping material attached to the inner surface of the waist band to prevent the waist band from slipping out of position during the therapy.

### CROSS-REFERENCE TO RELATED APPLICATION

This application claims the benefit of priority of U.S. provisional application No. 62/063,789, filed Oct. 14, 2014, the contents of which are herein incorporated by reference.

### BACKGROUND OF THE INVENTION

The present invention relates to a support member and, more particularly, to a support member that lifts the pannus during radiation therapy or other medical procedure.

When a patient with a large amount of abdominal fat is present for radiation therapy to the pelvic or groin region, the excess tissue must be lifted and immobilized away from the treatment area in a way which does not obstruct or interfere with the delivery of the treatment. Currently the only method to decrease skin folds and immobilize excessive abdominal tissue during pelvis radiation treatment is taping the tissue out of the way. Using tape to decrease skin folds and lift excessive tissue is not a reliable method for accurately reproducing daily treatment set-ups. Using tape is also painful to the patient and ineffective in eliminating all skin folds. Some patients are also allergic to tape. In addition to the technical problems, using tape is time consuming and may be embarrassing to the patient.

As can be seen, there is a need for a pannus support member that lifts and immobilizes excessive abdominal fat for radiation therapy and other medical procedures.

### SUMMARY OF THE INVENTION

In one aspect of the present invention, a pannus support member comprises: a waist band comprising a first end, a second end, an inner surface, and an outer surface, wherein the waist band is securable about an abdomen such that the pannus is contained and lifted by the waist band away from a target area on the abdomen; a pair of shoulder straps each comprising a first end and a second end secured to the waist band, wherein the pair of shoulder straps are adjustable in length; and a polymer gripping material attached to the inner surface of the waist band.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown in use;

FIG. 2 is a perspective view demonstrating a patient without the present invention;

FIG. 3 is a front perspective view of an embodiment of the present invention;

FIG. 4 is a rear perspective view of an embodiment of the present invention;

FIG. 5 is a perspective view of an embodiment of the present invention in a folded configuration;

FIG. 6 is a section detail view of the present invention taken along line 6-6 in FIG. 5;

FIG. 7 is a section detail view of the present invention taken along line 7-7 in FIG. 2; and

FIG. 8 is a section detail view of the present invention taken along line 8-8 in FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a belt that independently lifts and immobilizes excessive abdominal fat for the purpose of radiation therapy delivery without the use of bed rails or other supports which may interfere with the delivery of radiation therapy. This is possible because the present invention includes a wide stretchy belt which wraps tightly around the abdomen while adjustable shoulder straps lift and secure the belt in place. The present invention lifts excessive tissue gently out of the treatment area quickly and painlessly. The present invention enables the radiation therapist to more accurately reproduce patient set-ups and reduces the time it takes to set the patient up for their treatment. The present invention eliminates all or nearly all skin folds in the pelvic treatment area.

The present invention works by being wrapped around a supine patient's abdomen while the patient lifts any excessive abdominal tissue. The belt is closed tightly around the abdomen and adjustable shoulders straps are used to lift and hold the tissue in place. Once the radiation treatment is complete the belt is opened and removed.

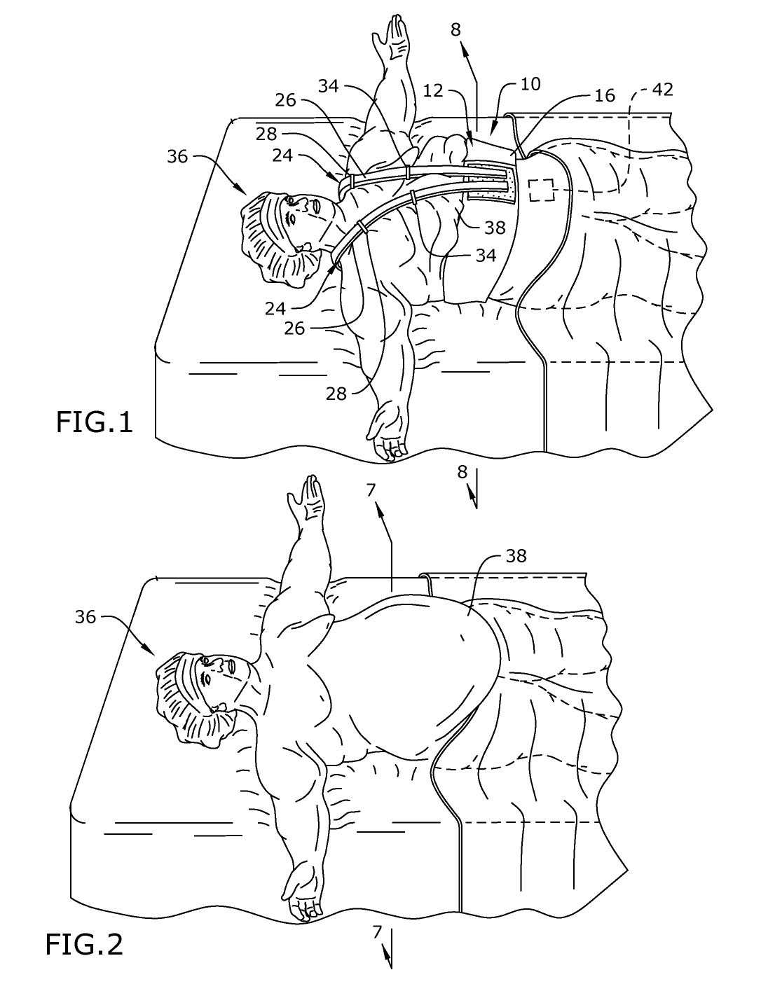

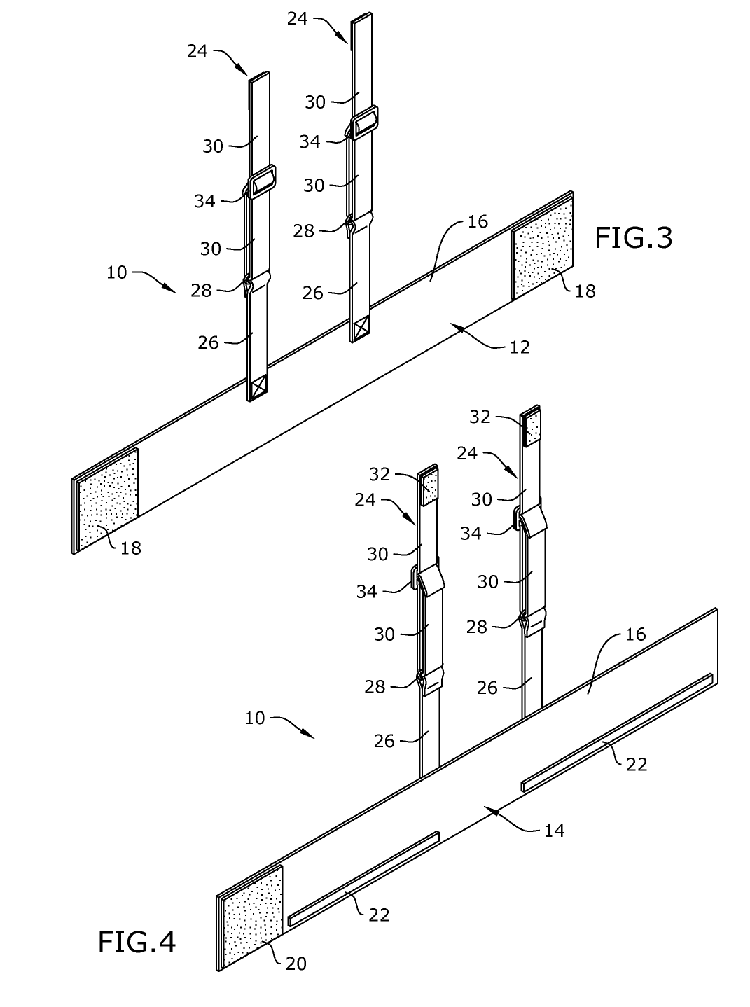

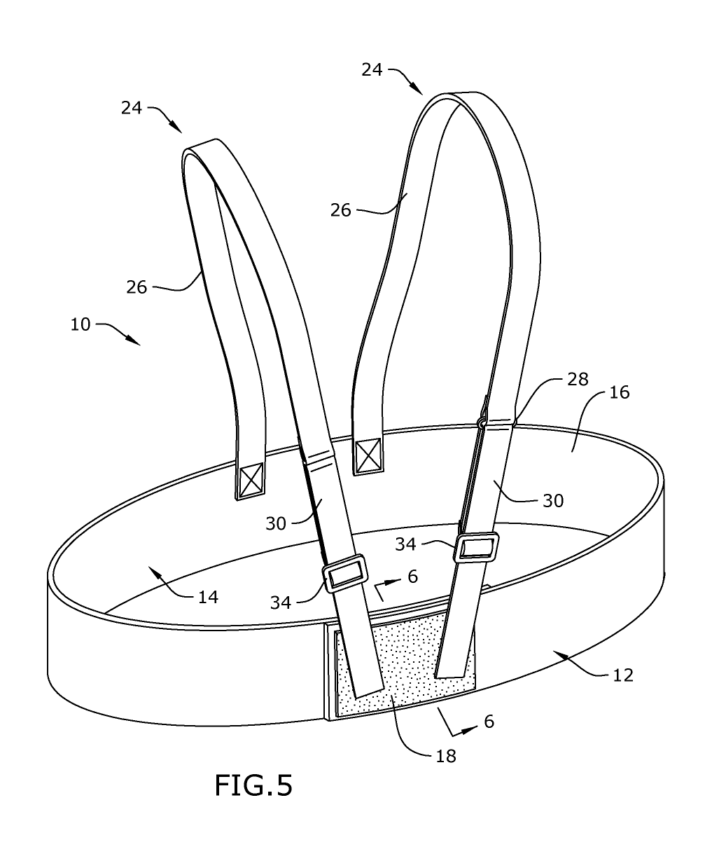

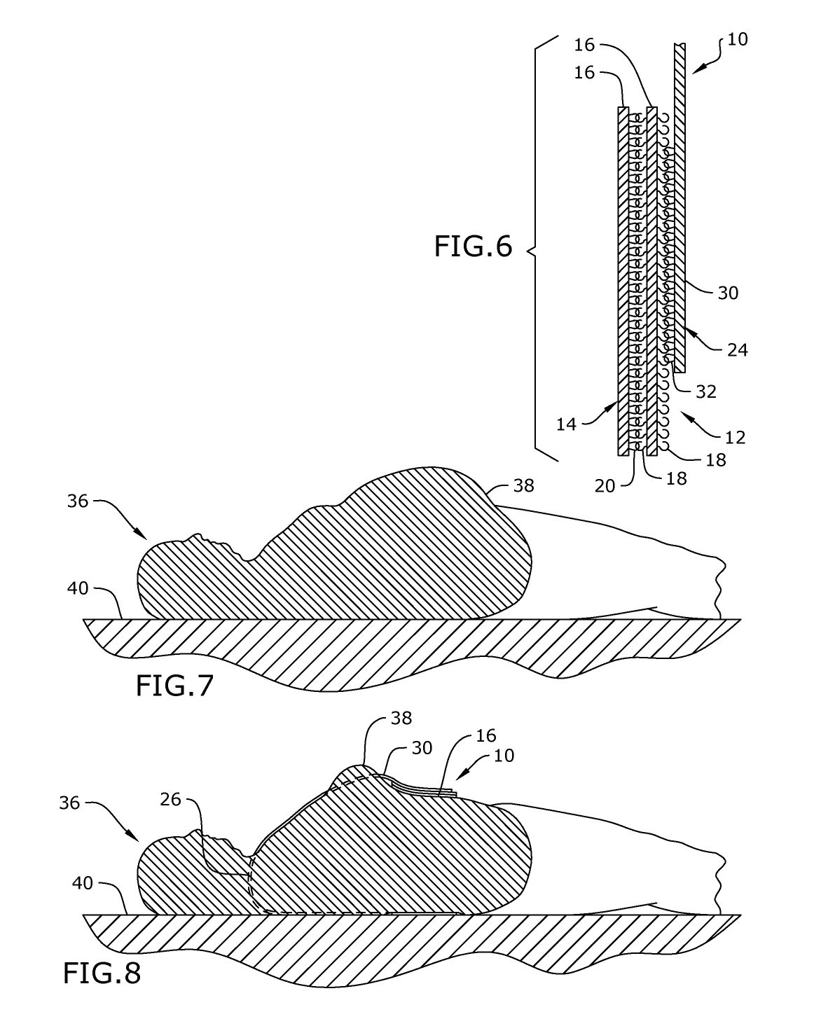

Referring to FIGS. 1 through 8, the present invention includes a pannus support member 10. The pannus support member 10 includes a waist band 16 having a first end, a second end, an inner surface 14 and an outer surface 12. The waist band 16 is securable about an abdomen such that the pannus 38 is contained and lifted by the waist band 16 away from a target area 42 on the abdomen of a patient 36. The present invention further includes a pair of shoulder straps 24 each having a first end and a second end secured to the waist band 16. The pair of shoulder straps 24 are adjustable in length, such that the straps 24 apply a lifting force to the waist band 16 to cause the pannus 38 to be maintained in a lifted position away from the target area 42. A polymer gripping material 22 is attached to the inner surface 14 of the waist band 16 to prevent the waist band 16 from slipping out of position during the therapy.

The waist band 16 of the present invention may include a wide elastic band that wraps around the patients abdomen. In certain embodiments, a first hook and loop patch 18 is attached to the outer surface 12 at the first end and a second hook and loop patch 18 is attached to the outer surface 12 at the second end. A third hook and loop patch 20 is secured to at least one of the first end and the second end of the inner surface 14. The waist band 16 may be wrapped around the abdomen, and one of the first hook and loop patch 18 and the second hook and loop patch 18 is releasably attached to the third hook and loop patch 20, thereby securing the waist band 16 to the patient 36.

The shoulder straps 24 include pair of straps being constructed to extend across shoulders of the patient 36. Each of the shoulder straps 24 may include an elastic portion 26 having a first end sewn to an external side 12 of the waist band 16. The second end of the elastic portion 26 may be sewn to a square ring 28. The shoulder straps 24 may further include webbing straps 30. The first end of the webbing straps 30 may be secured to the square ring 28 and the second end includes a fourth hook and loop patch 32. The webbing straps 30 may run through slide buckles 34, such as trip-glide buckles, which allow the shoulder straps 24 to be adjustable in length. The fourth hook and loop patch 32 releasably attaches to one of the first hook and loop patch 18 and the second hook and loop patch 18 when the waist band 16 is secured to the abdomen. The shoulder straps 24 may be tightened using the slide buckles, thereby apply the lifting force to the waist band 16.

The polymer gripping material 22 prevents the waist band 16 from slipping out of position during the therapy by gripping to the patient's skin. The polymer gripping material may be made of silicone and/or neoprene. However, the polymer gripping material is not limited to the above mentioned materials, and may be made of any non-slip materials. In certain embodiments, the polymer gripping material 22 may be formed into strips of material. The strips are attached to the inner surface 14 adjacent a bottom edge of the inner surface 14.

The present invention works by placing the open belt on a table, i.e. treatment table, CT table or simulation table. The shoulder straps are loosened and laying straight out. The patient is instructed to lay supine on top of the belt with the belt directly behind the patient's back. The patient then lifts their abdomen pulling excessive tissue superiorly. After the abdomen has been lifted, the belt is wrapped tightly around the abdomen under the lifted excess abdominal tissue. Usually this may require two therapists, one for each end of the belt. The belt is then secured with the hook and loop fasteners at the ends of the belt. The shoulder straps are then pulled over the shoulders and secured to the front of the belt essentially holding the excess fat in place. The shoulder straps can be adjusted for a custom fit. The present invention could possibly be used to lift the excessive abdominal tissue of patients for other radiologic technologies such as ultrasound. The present invention may also have potential in other medical modalities that may require the abduction of excessive abdominal tissues.

A method of making the present invention may include the following. A wide long piece of stretchy tension controlled material, such as elastic material, is used to form the main body of the belt. A strip of gripping material, such as neoprene material, may be sewn along the inside inferior portion of the elastic band. An area of loop type fastening material is sewn to the inside portion of one end of the belt. Next an area of hook type fastening material is sewn on both outside end portions of the belt. To make the shoulder straps, a loop fastening material to the distal ends of two long lengths of webbing material that are about 2 inches wide. This section of the shoulder strap is then threaded through a plastic rectangle ring and tri-glide buckle and sewn to the tri-glide buckle in such a way as to allow for adjustment of the webbing portion of the shoulder strap. A second portion of the shoulder strap is made using an elastic strap the same width as the webbing portion of the strap and sewing the end of this strap to the rectangle ring. This will attach the two portions of the straps making one long adjustable shoulder strap. The shoulder strap is then sewn to the posterior of the belt with the elastic portion to the back of the belt and the webbing portion at the front of the belt.

It should be understood, of course, that the foregoing relates to exemplary embodiments of the invention and that modifications may be made without departing from the spirit and scope of the invention as set forth in the following claims.

What is claimed is:

1. A pannus support member comprising:
a waist band comprising a first end, a second end, an inner surface, and an outer surface, wherein the waist band is securable about an abdomen such that the pannus is contained and lifted by the waist band away from a target area on the abdomen;
a pair of shoulder straps each comprising a first end and a second end secured to the waist band, wherein the pair of shoulder straps are adjustable in length; and
a polymer gripping material attached to the inner surface of the waist band.

2. The pannus support member of claim 1, further comprising a first hook and loop patch attached to the outer surface at the first end, a second hook and loop patch attached to the outer surface at the second end, and a third hook and loop patch secured to one of the first end and the second end of the inner surface, wherein one of the first hook and loop patch and the second hook and loop patch is releasably attached to the third hook and loop patch when secured to the abdomen.

3. The pannus support member of claim 2, wherein the first end of each of the pair of shoulder straps is sewn to the waist band, and the second end of each of the pair of shoulder straps comprises a fourth hook and loop patch releasably attached one of the first hook and loop patch and the second hook and loop patch when the waist band is secured to the abdomen.

4. The pannus support member of claim 1, wherein the waist band is formed of an elastic material.

5. The pannus support member of claim 1, wherein at least a portion of the pair of shoulder straps is formed of an elastic material.

6. The pannus support member of claim 1, wherein the polymer gripping material is one of silicone and neoprene.

7. The pannus support member of claim 1, wherein the polymer gripping material comprises a strip of material attached to the inner surface adjacent a bottom edge of the inner surface.

8. The pannus suppot member of claim 1, wherein the pair of shoulder straps are each adjustable by a slid buckle.
